(19) United States
(12) Patent Application Publication
(10) Pub. No.: US 2004/0009589 A1
(43) Pub. Date: Jan. 15, 2004

(54) Endothelial cells derived from human embryonic stem cells

(76) Inventors: Shulamit Levenberg, Brighton, MA (US); Michal Amit, Misgav (IL); Joseph Itskovitz-Eldor, Haifa (IL); Robert Langer, Newton, MA (US)

Correspondence Address: Valarie B. Rosen, Choate, Hall & Stewart, 53 State Street, Exchange Place, Boston, MA, 02109, US

(21) Appl. No.: 10/396,082

(22) Filed: Mar. 25, 2003

Related U.S. Application Data
(60) Provisional application No. 60/367,689, filed on Mar. 26, 2002.

(51) Int. Cl. C12N 5/08

(52) U.S. Cl. 435/366

(57) ABSTRACT

The invention is a population of embryonic endothelial cells produced in vitro from human embryonic stem cells. The cells produce platelet endothelial cell adhesion molecule-1 and are vasculogenic. The cells may be combined with a cell support substrate, seeded on a polymer matrix, or combined with a cell-support substrate that is infused into a polymer matrix. The cells may also be injected directly into a tissue site.

This application claims priority from U.S. Provisional Application No. 60/367,689, filed Mar. 26, 2002, the entire contents of which are incorporated herein by reference.

### FIELD OF THE INVENTION

This invention pertains to the use of embryonic stem cells, and, more specifically, to the differentiation, isolation, characterization and use of human embryonic endothelial cells.

### BACKGROUND OF THE INVENTION

Human vascular endothelial cells are important for developing engineered vessels for treatment of vascular disease and may also be useful for augmenting vessel growth to areas of ischemic tissue or following implantation (Niklason, et al., (1999) Science 284, 489-93; Kawamoto, et al., (2001) Circulation 103, 634-7). Endothelial progenitor cells from adults have vasculogenic potential (Kawamoto, 2001). Vasculogenesis is defined as the in situ assembly of capillaries from undifferentiated endothelial cells, as opposed to angiogenesis, the sprouting of capillaries from preexisting blood vessels (Yancopoulos, et al., (1998) Cell 93, 661-4). This potential can be exploited in tissue engineering for induction of tissue vascularization, especially for complex tissues where vascularization of regenerating tissue is essential. For example, it is often desirable to vascularize engineered tissue in vitro prior to transplantation (Black, et al., (1998) FASEB J 12, 1331-40; Kaihara, et al., (2000) Tissue Eng 6, 105-17). Vascularization in vitro is important to enable cell viability during tissue growth, induce structural organization and promote integration upon implantation. The use of embryonic stem cells in tissue engineering and other applications in place of adult endothelial progenitor or endothelial cells would be particularly exciting, since ES cells can be expanded without apparent limit and ES cell-derived cells could be created in virtually unlimited amounts and available for potential clinical use (Amit, et al., (2000) Dev Biol 227, 271-8).

The vasculogenic potential of the embryonic cells could specifically be of use in tissue engineering for induction of tissue vascularization. A potential source of cells for these applications are embryonic stem cells which, in murine systems, were shown to differentiate into endothelial cells forming vascular structures in a process called vasculogenesis (Vittet, et al., (1996) Blood 88, 3424-31). Early endothelial progenitor cells isolated from differentiating mouse embryonic stem cells were shown to give rise to three blood vessel cell components, hematopoetic, endothelial and smooth muscle cells (Yamashita, et al., (2000) Nature 408, 92-6). Therefore, in addition to potential clinical applications, purified human embryonic endothelial cells could be important for studying early human development and differentiation of embryonic stem cells into various tissues.

Differentiation of embryonic stem cells into endothelial cells and formation of vessel structure has been studied extensively in murine embryogenesis, including maturation steps, molecular events and growth factor involvement (Keller, G. M. (1995) Curr Opin Cell Biol 7, 862-9; Hirashima, et al., (1999) Blood 93, 1253-63). However, lack of experimental cell systems, had made it difficult to study these developmental processes in the human until now. Human embryonic stem cell lines (hES) recently established from the inner cell mass of human blastocytes provide a unique system for studying these events in human embryonic development (Thomson, et al., (1998) Science 282, 1145-7). Human ES cells have the potential to generate all embryonic cell lineages when they undergo differentiation. Differentiation of hES can be induced by removing the cells from their feeder layer and growing them in suspension. This differentiation in suspension, results in aggregation of the cells and formation of embryoid bodies (EBs) in which successive differentiation steps occur (Itskovitz-Eldor, et al., (2000) Mol Med 6, 88-95).

### SUMMARY OF THE INVENTION

The invention uses a population of human embryonic endothelial cells produced in vitro from human embryonic stem cells. The cells may be vasculogenic. Alternatively, or in addition, the cells express one or more of PECAM1, GATA-2, N-cadherin, VE-cadherin, VWF, and CD34. The cells may incorporate ac-LDL. In one embodiment, a tissue engineering construct is formed by combining the human embryonic endothelial cells with a cell support substrate. A polymer matrix may be infused with the cell support substrate. The polymer matrix may have any shape, for example, particles, tube, sponge, sphere, strand, coiled strand, capillary network, film, fiber, mesh, or sheet. A growth factor may be attached to the polymer matrix or combined with the cell support substrate. The cell support substrate may be a gel and may be combined with a liquid carrier, for example, phosphate buffered saline. The gel may be Matrigel™ or a collagen-GAG gel. Alternatively, the gel may include one or more of collagen I, collagen IV, laminin, fibrin, fibronectin, proteoglycans, glycoproteins, glycoaminoglycans, proteinases, collagenases, chemotactic agents, or growth factors. An additional cell type may be combined with the human embryonic endothelial cells in the tissue engineering construct. For example, such cells may be muscle cells, nerve cells, connective tissue cells, or stem cells. The cell-support substrate may be a tube, for example, a decellularized blood vessel, a synthetic polymer tube, or a collagen tube, in which the cells are disposed on an inner surface.

In another aspect, the invention provides a method of producing vasculogenic human cells in vitro. The method includes providing a population of human embryonic stem cells, culturing the stem cells in the absence of both LIF and bFGF to stimulate formation of embryoid bodies containing the cultured stem cells, and isolating PECAM1 positive cells from the embryoid bodies. The step of isolating may include dissociating the embryoid bodies to separate the cultured stem cells, incubating the cultured stem cells with a labeled PECAM1 antibody to distinguish the portion of the cultured stem cells that are PECAM1+, and separating the PECAM1+ cells from the remaining cultured stem cells. The step of providing may include incubating a population of human embryonic stem cells in a culture medium and at least partially disaggregating the stem cells. The vasculogenic human cells produced by this method may be suspended in or on a liquid carrier, a cell-support substrate, or a mixture of both, and delivered to a tissue in an animal. Alternatively, the vasculogenic cells may be deposited on a polymer matrix by infusing the matrix with the cell suspension (either with or without the cell support substrate). The cell suspension may include an additional cell type, or the additional cell type may be added separately to the polymer matrix. The polymer matrix may be delivered to a tissue site. For example, the polymer matrix may be disposed about the outside of a blood vessel. The cells may be allowed to proliferate within the cell support substrate or on the polymer matrix before being delivered to a tissue site. A mechanical force may be imparted on the cells during proliferation. The mechanical force may be cyclic. Any force is appropriate, for example, a hoop stress, a shear stress, a hydrostatic stress, a compressive stress, or a tensile stress. The cells may be delivered to any type of tissue, for example, connective tissue, muscle tissue, nerve tissue, or organ tissue. The cells may form a vascular structure during proliferation.

The numerical values herein include a range of values whose boundaries are defined by the limits of precision and accuracy of the applicable measurement technique and rounding of numbers during calculations.

### BRIEF DESCRIPTION OF THE DRAWING

The file of this patent contains at least one drawing executed in color. Copies of this patent with color drawings will be provided by the Office upon request and payment of the necessary fee.

The invention is described with reference to the several figures of the drawing, in which,

FIG. 1. Endothelial gene expression in hES-derived EBs by RT-PCR analysis. (A) RNA was isolated from undifferentiated hES cells and from hEBs at different time points (days) during differentiation, and subjected to RT-PCR analysis. The negative controls, no template (N.T.) and MEF, and the HUVEC positive control (HUV) are shown to the right. (B) Quantitative analysis of gene expression. Relative pixel intensity corresponds to gene expression level; for each time point, mean pixel intensities of each band were measured and normalized to mean pixel intensities of GAPDH band. The results shown are mean values of three different experiments, plus and minus standard deviation.

FIG. 2. Expression of endothelial cell markers in vessel-like structure within hEBs. (A) EBs at day 13 stained with human PECAM1 antibodies (Red), von Willebrand Factor antibodies (Green) and DAPI for nuclear staining (Blue). PECAM1 is organized at cell-cell junctions while VWF is found in organelles in the cytoplasm. (B) EB cells stained with human VE-cadherin antibodies (Red) and DAPI (Blue). (Orig. mag. X1000). (C) Low magnification (X100) of EB stained with PECAM1 antibodies. (D) Areas of PECAM1 positive cells (Red) within part of an EB, organized in elongated clusters. Cells nuclei stained with DAPI (Blue). (orig. mag. X400). (E) Channels forming PECAM1 positive cells within a 13-day-old EB (orig. mag. X200).

FIG. 3. Confocal microscopy of EBs stained for PECAM1, showing three dimensional network formations, vascular-like channels. (A) 4-day-old EB, (B) 6-day-old EB, (C) 10-day-old EB and (D) 13-day-old EB. Notice the intensive and complicated vascular network developed at day 10-13 old EBs. (orig. mag. X100).

FIG. 4. Isolation of endothelial cells from human embryoid bodies using fluorescent-labeled anti PECAM1 antibodies and analysis of the sorted cells. (A) EBs at day 13 were dissociated and incubated with PECAM1 antibodies. Fluorescent-labeled cells were isolated using a flow cytometry cell sorter. (B) Flow cytometric analysis of endothelial cell markers in PECAM1+ cells grown in culture for 6 passages and HUVEC cells. The cells were dissociated and incubated with either isotype control (dashed lines) or antigen specific antibodies as indicated (Solid lines). Percent positive cells are shown.

FIG. 5. Characterization of hES-derived endothelial cells grown in culture. (A) Immunofluorescence staining of PECAM1 (red) at cell-cell junctions and vWE (green) in the cytoplasm. The nuclei are stained with DAPI (blue). Lower magnification (X200) of the cells stained for PECAM1 is shown in (B). (C) N-cadherin and (D) VE-cadherin staining, in cell-cell adherent junctions. (E) Double staining for Vinculin (red) and Actin (green). Vinculin is found in both focal contacts and cell-cell adherent junctions where it associates with actin stress fibers ends. (Orig. mag. for A and C-E X1000) (F) Uptake of Dill-labeled ac-LDL by PECAM1+ cells. (G-H) Cords formation by PECAM1+ cells 24 hours (G) or 3 days (H) after seeding the cells in Matrigel. (Orig. mag for G=X100 and for H=X200). (I) Electron microscopy of the cord cross-section showing lumen formation (Bar=2 μm) and (J) higher magnification of the lumen (lu) area showing cell-cell interactions closing the lumen and the nucleus (n) of one cell (Bar=8 μm).

FIG. 6. Transplantation of embryonic endothelial cells (PECAM1+) in SCID mice. PECAM1+ cells were seeded onto PLLA/PLGA polymer scaffolds as described in Materials and Methods. The cells+scaffolds were implanted subcutaneously in the dorsal region of 4 weeks old SCID mice. (A-C) Immunoperoxidase (brown) staining of 7 day implants with anti human PECAM1 antibodies and (D-E) of 14 day implants with anti human CD34 antibodies, showing microvessels that are immunoreactive with these human-specific antibodies. Some of these human-positive microvessels have mouse blood cells in their lumen. (orig. mag. X400).

### DETAILED DESCRIPTION OF CERTAIN PREFERRED EMBODIMENTS

The isolation of human embryonic endothelial cells has potential therapeutic implications including cell transplantation for repair of ischemic tissues and tissue engineering of vascular grafts. Recently, several studies demonstrated the use of adult endothelial progenitor cells for such applications (Kawamoto, 2001; Kaushal, et al., (2001) Nat Med 7, 1035-40). Another source of cells for these applications are embryonic stem cells which, in murine systems, were shown to differentiate into endothelial cells forming vascular structures through vasculogenesis (Vittet, et al., (1996) Blood 88, 3424-31). Early endothelial progenitor cells isolated from differentiating mouse embryonic stem cells were shown to give rise to three blood vessel cell components, hematopoetic, endothelial and smooth muscle cells (Yamashita, et al., (2000) Nature 408, 92-6). In addition, it was recently shown that endothelial progenitors and embryonic endothelial cells could differentiate into beating cardiomyocytes when cocultured with neonatal cardiomyocytes or when injected near a damaged heart area (Condorelli, G., et al. (2001) Proc. Natl. Acad. Sci. USA 98, 10733-10738). It also has been shown that embryonic endothelial cells are critical for the earliest stages of liver and pancreas organogenesis (Matsumoto, K., et al. (2001) Science 294, 559-563; Lammert, E., et al. (2001) Science 294, 564-567). Since the formation of the first capillaries takes place mostly during early stages of embryogenesis when endothelial cells are generated from precursor cells, isolated human embryonic endothelial cells or progenitor cells can be important for such applications (Flamme, et al., (1997) J Cell Physiol 173, 206-10). Therefore, in addition to potential clinical applications, purified human embryonic endothelial cells could be important for studying early human development and differentiation of embryonic stem cells into various tissues.

Differentiation of embryonic stem cells into endothelial cells and formation of vessel structure has been studied extensively in murine embryogenesis, including maturation steps, molecular events and growth factor involvement (Keller, G. M. (1995) Curr Opin Cell Biol 7, 862-9; Hirashima, et al., (1999) Blood 93, 1253-63). However, lack of experimental cell systems, had made it difficult to study these developmental processes in the human until now. Human embryonic stem cell lines (hES) recently established from the inner cell mass of human blastocytes provide a unique system for studying these events in human embryonic development (Thomson, et al., (1998) Science 282, 1145-7). Human ES cells have the potential to generate all embryonic cell lineages when they undergo differentiation. Differentiation of hES can be induced by removing the cells from their feeder layer and growing them in suspension. This differentiation in suspension, results in aggregation of the cells and formation of embryoid bodies (EBs) in which successive differentiation steps occur (Itskovitz-Eldor, et al., (2000) Mol Med 6, 88-95).

In one embodiment, the invention is a population of human embryonic endothelial cells. The cells may be produced by culturing human embryonic stem cells in the absence of LIF and bFGF to stimulate formation of embryoid bodies, and isolating PECAM1 positive cells from the population. Using techniques described herein, we show an increase in expression of several endothelial cell-specific genes during EB differentiation reaching a maximum between days 13-15, and development of extensive vasculature-resembling structures within the EB. We isolated human embryonic endothelial cells from day 13-15 EBs using platelet endothelial cell adhesion molecule-1 (PECAM1) antibodies and characterized their behavior in vitro and in vivo.

In one embodiment, cells produced according to the techniques provided by the invention express PECAM1, transcription factor GATA-2, N-cadherin, vascular endothelial-cadherin and von Willebrand factor. For example, at least 45%, in a further example, 55% or 65%, express at least one of these proteins. In a further example, at least 75%, at least 85%, or at least 95% of the cells may express one or more of these proteins. Alternatively or in addition, at least 45%, for example, at least 55%, or at least 65% may incorporate ac-LDL (acetylated low density lipoprotein). In a further example, at least 75%, at least 85%, or at least 95% of the cells may incorporate ac-LDL. Alternatively or in addition, at least 10%, for example, at least 12% or at least 14% of the cells may express CD34. In a further example, at least 16%, at least 18%, or at least 20% of the cells may express CD34. As used herein, the term “expression” indicates that the cell produces an mRNA transcript of a particular gene or a protein translated from that transcript.

These cells may be combined with a cell support substrate including extracellular matrix components. The substrate may be a gel, for example, Matrigel™, from Becton-Dickinson. Matrigel™ is a solubilized basement membrane matrix extracted from the EHS mouse tumor (Kleinman, H. K., et al., Biochem. 25:312, 1986). The primary components of the matrix are laminin, collagen I, entactin, and heparan sulfate proteoglycan (perlecan) (Vukicevic, S., et al., Exp. Cell Res. 202:1, 1992). Matrigel™ also contains growth factors, matrix metalloproteinases (MMPs [collagenases]), and other proteinases (plasminogen activators [PAs]) (Mackay, A. R., et al., BioTechniques 15:1048, 1993). The matrix also includes several undefined compounds (Kleinman, H. K., et al., Biochem. 25:312, 1986; McGuire, P. G. and Seeds, N. W., J Cell. Biochem. 40:215, 1989), but it does not contain any detectable levels of tissue inhibitors of metalloproteinases (TIMPs) (Mackay, 1993).

In another embodiment, the gel may be a collagen I gel. Such a gel may also include other extracellular matrix components, such as glycosaminoglycans, fibrin, fibronectin, proteoglycans, and glycoproteins. The gel may also include basement membrane components such as collagen IV and laminin. Enzymes such as proteinases and collagenases may be added to the gel, as may cell response modifiers such as growth factors and chemotactic agents.

The cells, either mixed with a gel or simply with a liquid carrier such as PBS, may be injected directly into a tissue site where vasculogenesis is desired. For example, the cells may be injected into ischemic tissue in the heart or other muscle, where the cells will organize into tubules that will anastamose with existing cardiac vasculature to provide a blood supply to the diseased tissue. Other tissues may be vascularized in the same manner. The cells will incorporate into neovascularization sites in the ischemic tissue and accelerate vascular development and anastamosis (see Kawamoto, 2001). It is intended that the invention be used to vascularize all sorts of tissues, including connective tissue, muscle tissue, nerve tissue, and organ tissue. Non-blood duct networks may be found in many organs, such as the liver and pancreas, and the techniques of the invention may be used to engineer or promote healing in such tissues as well. For example, embryonic endothelial cells injected into the liver can develop into tubular networks around which native hepatocytes can develop other liver structures.

The embryonic endothelial cells may also be used to help heal cardiac vasculature following angioplasty. For example, a catheter can be used to deliver embryonic endothelial cells to the surface of a blood vessel following angioplasty or before insertion of a stent. Alternatively, the stent may be seeded with embryonic endothelial cells. Blood vessels treated with adult endothelial cells exhibit accelerated re-endothelialization, preventing restenosis in the injured vessel (Parikh, et al. (2000) Advanced Drug Delivery Reviews, 42, 139-161). In another embodiment, embryonic endothelial cells may be seeded into a polymeric sheet and wrapped around the outside of a blood vessel that has undergone angioplasty or stent insertion (Nugent, et al. (2001) J Surg. Res., 99, 228-234). The cells may also be mixed with a gel and infused into the polymer sheet instead of directly seeded onto the matrix.

If a stiffer implant is desired, the cells may be seeded onto a polymer matrix, for example, a sponge, which is then implanted into the desired tissue site. Alternatively, the cells may be mixed with a gel which is then absorbed onto the interior and exterior surfaces of the matrix and which may fill some of the pores of a spongy or other porous matrix. Capillary forces will retain the gel on the matrix before hardening, or the gel may be allowed to harden on the matrix to become more self-supporting.

Preferably, the polymer matrix is biodegradable. Suitable biodegradable matrices are well known in the art and include collagen-GAG, collagen, fibrin, PLA, PGA, and PLA-PGA co-polymers. Additional biodegradable materials include poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes and polysaccharides. Non-biodegradable polymers may also be used as well. Other non-biodegradable, yet biocompatible polymers include polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, and poly(ethylene oxide). Those skilled in the art will recognize that this is an exemplary, not a comprehensive, list of polymers appropriate for tissue engineering applications.

It is preferred that the matrix be formed with a microstructure similar to that of the extracellular matrix that is being replaced. Mechanical forces imposed on the matrix by the surrounding tissue will influence the cells on the artificial matrix and promote the regeneration of extracellular matrix with the proper microstructure. The cross-link density of the matrix may also be regulated to control both the mechanical properties of the matrix and the degradation rate (for degradable scaffolds). The shape and size of the final implant should be adapted for the implant site and tissue type. The matrix may serve simply as a delivery vehicle for the cells or may provide a structural or mechanical function. The matrix may be formed in any shape, for example, as particles, a sponge, a tube, a sphere, a strand, a coiled strand, a capillary network, a film, a fiber, a mesh, or a sheet.

PLA, PGA and PLA/PGA copolymers are particularly useful for forming the biodegradable matrices. PLA polymers are usually prepared from the cyclic esters of lactic acids. Both L(+) and D(−) forms of lactic acid can be used to prepare the PLA polymers, as well as the optically inactive DL-lactic acid mixture of D(−) and L(+) lactic acids. PGA is the homopolymer of glycolic acid (hydroxyacetic acid). In the conversion of glycolic acid to poly(glycolic acid), glycolic acid is initially reacted with itself to form the cyclic ester glycolide, which in the presence of heat and a catalyst is converted to a high molecular weight linear-chain polymer. The erosion of the polyester matrix is related to the molecular weights. The higher molecular weights, weight average molecular weights of 90,000 or higher, result in polymer matrices which retain their structural integrity for longer periods of time; while lower molecular weights, weight average molecular weights of 30,000 or less, result in both slower release and shorter matrix lives. For example, poly(lactide-co-glycolide) (50:50) degrades in about six weeks following implantation.

In an exemplary embodiment, a cell response modifier such as a growth factor or a chemotactic agent may be added to the polymer matrix. Such a modifier, for example, vascular endothelial-derived growth factor, may be used to promote differentiation of the embryonic endothelial cells. Alternatively, the modifier may be selected to recruit cells to the matrix or to promote or inhibit specific metabolic activities of cells recruited to the matrix. Exemplary growth factors include epidermal growth factor, bone morphogenetic protein, TGFβ, hepatocyte growth factor, platelet-derived growth factor, TGFα, IGF-I and II, hematopoetic growth factors, heparin binding growth factor, peptide growth factors, and basic and acidic fibroblast growth factors. In some embodiments it may be growth factors such as nerve growth factor (NGF) or muscle morphogenic factor (MMP). The particular growth factor employed should be appropriate to the desired cell activity. The regulatory effects of a large family of growth factors are well known to those skilled in the art.

The cell-seeded polymer matrix, with or without the gel, may be implanted into any tissue, including connective, muscle, nerve, and organ tissues. For example, an implant placed into a bony defect will attract cells from the surrounding bone which will synthesize extracellular matrix, while the embryonic endothelial cells form blood vessels. The blood supply for the new bone will be provided as the new ECM is formed and mineralized. An implant placed into a skin defect will promote dermis formation and provide a vascular network to supply nutrients to the newly formed skin.

Alternatively, the cells may be seeded onto a tubular substrate. For example, the polymer matrix may be formed into a tube or network. Such tubes may be formed of natural or synthetic ECM materials such as PLA or collagen or may come from natural sources, for example, decellularized tubular grafts. The embryonic endothelial cells will coat the inside of the tube, forming an artificial channel that can be used for a heart bypass. In addition, use of embryonic endothelial cells may reduce thrombosis post-implantation (see Kaushall, 2001).

The cells may be allowed to proliferate on the polymer matrix or tubular substrate before being implanted in an animal. During proliferation, mechanical forces may be imposed on the implant to stimulate particular cell responses or to simulate the mechanical forces the implant will experience in the animal. For example, a medium may be circulated through a tubular substrate in a pulsatile manner (i.e., a hoop stress) or with sufficient speed to exert a sheer stress on cells coating the inside of the tube (Niklason, 1999; Kaushal, 2001). Alternatively, a hydrostatic force or compressive force may be imparted on an implant that will be deposited within an organ such as the liver, or a tensile stress may be imparted on an implant that will be used in a tissue that experiences tensile forces.

Cells that are recruited to the implant may also differentiate into other cell types. Bone cell precursors migrating into a bone implant can differentiate into osteoblasts. Mesenchymal stem cells migrating into a blood vessel can differentiate into muscle cells. Endothelial cells forming tubular networks in liver can induce the formation of liver tissue.

In another embodiment, the embryonic endothelial cells are mixed with another cell type before implantation. The cell mixture may be suspended in a carrier such as a culture medium or in a gel as described above. Alternatively, the cells may be co-seeded onto a polymer matrix or combined with a gel that is absorbed into the matrix. While cumbersome, it may be desirable to seed one cell type directly onto the matrix and add the second cell type via a gel. Any ratio of embryonic endothelial cells to the other cell type or types may be used. One skilled in the art will recognize that this ratio may be easily optimized for a particular application. Exemplary ratios of embryonic endothelial cells to other cells are at least 10% (e.g., 1:9), at least 25%, at least 50% (e.g., 1:1), at least 75%, and at least 90%. Smaller ratios, for example, less than 10%, may also be employed.

Any cell type, including connective tissue cells, nerve cells, muscle cells, organ cells, or other stem cells, may be combined with the embryonic endothelial cells. For example, osteoblasts may be combined with the embryonic endothelial cells to promote the co-production of bone and its vasculature in a large defect. Fibroblasts combined with embryonic endothelial cells and inserted into skin will produce fully vascularized dermis. Other exemplary cells that may be combined with the embryonic endothelial cells of the invention include ligament cells, lung cells, epithelial cells, smooth muscle cells, cardiac muscle cells, skeletal muscle cells, islet cells, nerve cells, hepatocytes, kidney cells, bladder cells, and bone-forming cells.

Materials and Methods

Cell culture. hES cells (H9 clone), were grown on mouse embryo fibroblasts (Cell Essential) in KnockOut Medium (Gibco-BRL, Gaithersburg, Md.), a modified version of Dulbeco's modified Eagle's medium optimized for ES cells (Itskovitz-Eldor, et. al., (2000) Mol. Med. 6, 88-95, the contents of which are incorporated herein by reference). Tissue cover plates were covered with 0.1% gelatin (Sigma). Culture were grown in 5% CO₂ and were routinely passaged every 5-6 days after disaggregating with 1 mg/ml collagenase type IV (Gibco-BRL). To induce formation of EBs, hES colonies were digested using either 1 mg/ml collagenase type IV or trypsin/EDTA (0.1%/1 mM) and transferred to petri dishes to allow their aggregation and prevent adherence to the plate. Human EBs were grown in the same culture medium without LIF and bFGF. Isolated PECAM1+ cells were grown on plates coated with 1% gelatin in endothelial growth medium, EGM-2 (Clonetics,) and passaged using 0.025%/0.01% trypsin/EDTA (Clonetics). HUVEC cells (Clonetics) were grown on regular tissue culture plates in EGM-2 medium. For Matrigel differentiation assay, cells removed from confluent culture by trypsin treatment were seeded in Matrigel-coated 35 mm plates (BD Biosciences) at a concentration of 1×10⁵ cells per 300 μl of culture medium. After 30 min of incubation at 37° C., 1 ml of medium was added. Cord formation was evaluated by contrast-phase microscopy 24 hours or 3 days after seeding the cells.

Reverse Transcription (RT)-PCR Analysis.

Total RNAs from undifferentiating hES cells and from EBs were isolated using RNEasy Mini Kit (Qiagen). RT-PCR reaction was performed by using Qiagen OneStep RT-PCR kit with addition of 10 units Rnase inhibitor (Gibco-BRL) and with 40 ng RNA. To ensure semi quantitative results of the RT-PCR assays the number of PCR cycles for each set of primers was checked to be in the linear range of the amplification. In addition all RNA samples were adjusted to yield equal amplification of GAPDH as an internal standard. Primer sequences, reaction conditions and optimal cycle numbers are as follows:

Reverse Transcription (RT)-PCR Conditions

| | |
|---|---|
| RT and activation | 30° C. for 30 min, 95° C. for 15 min. |
| Amplification | 94° C. for 1 min, 55° C. for 1 min, 72° C. for 1 min. See below for cycle numbers. |
| Final extension | 72° C. for 10 min. |

Optimal Cycle Numbers

| | |
|---|---|
| PECAM1 | 28 cycles |
| VE-cad | 32 cycles |
| CD34 | 30 cycles |
| Flk-1 | 31 cycles |
| Tie-2 | 31 cycles |
| GATA-2 | 33 cycles |
| GATA-3 | 29 cycles |
| AC133 | 27 cycles |
| OCT-4 | 31 cycles |
| GAPDH | 24 cycles |

Optimal cycle numbers were determined for each gene to ensure that conditions were in the linear range
of PCR amplification

Primer Sequences

| | |
|---|---|
| PECAM1 | GCTGTTGGTGGAAGGAGTGC/GAAGTTGGCTGGAGGTGCTC |
| VE-cad | CCGGCGCCAAAAGAGAGA/CTGGTTTTCCTTCAGCTGGAAGTGGT |
| CD34 | TGAAGCCTAGCCTGTCACCT/CGCACAGCTGGAGGTCTTAT |
| Flk-1 | CAACAAAGCGGAGAGGAG/ATGACGATGGACAAGTACCC |
| Tie-2 | CCTTAGTGACATTCTTCC/GCAAAAATGTCCACCTGG |
| GATA-2 | CCCTAAGCAGCGCAGCAAGAC/TGACTTCTCCTGCATGCACT |
| GATA-3 | ACCCCACTGTGGCGGCGAGAT/CACAGCACTAGAGACC |
| AC133 | CAGTCTGACCAGCGTGAAAA/GGCCATCCAAATCTGTCCTA |
| OCT-4 | GAGAACAATGAGAACCTTCAGGAGA/TTCTGGCGCCGGTTACAGAACCA |
| GAPDH | AGCCACATCGCTCAGACACC/GTACTCAGCGGCCAGCATCG |

The amplified products were separated on 1.2% agarose gels with ethidium bromide (E-Ge1, Invitrogen). For each time point, mean pixel intensities of each band were measured and normalized to mean pixel intensities of GAPDH band. The values for three experiments were then averaged and graphed with standard deviation.

Immunochemical Reagents and Procedures.

For staining, EBs were transferred to gelatin-coated cover slips with medium containing 10% FBS. EBs, following attachment to the cover slips, or cells grown on gelatin-coated cover slips were fixed with methanol for 5 min at −20° C. or with 3% paraformaldehyde at room temperature and stained for 30 min with the relevant primary antibodies: anti-human PECAM1, anti-human vinculin (Sigma), anti-human von Willebrand factor (vWF) (Dako), purified monoclonal anti-N-cadherin and anti-human VE-cad (7B4) (Volk, et al., (1986) J Cell Biol 103, 1451-64; Lampugnani, et al., (1992) J Cell Biol 118, 1511-22). The secondary antibodies were Cy3-labeled goat anti mouse IgG (Jackson Laboratories) and Alexa Fluor goat anti rabbit IgG (Molecular Probes). In some cases cells or EBs were also stained with DAPI and FITC-phalloidin (Sigma). Following the indirect immunolabeling, cells were mounted in Floromount-G (Southern Biotechnology) and were examined using either a conventional fluorescence microscope (Nikon) or Ziess LSM 510 confocal microscope.

For uptake of Dill-labeled ac-LDL, PECAM1+ cells and control PECAM-cells were incubated with 10 μg/ml Dill-labeled ac-LDL (Biomedical Technologies Inc) for 4 h at 37° C. Following incubation, cells were washed 3 times with PBS, fixed with 3% paraformaldehyde for 30 minutes and visualized using a fluorescent microscope (Nikon).

For immunohistology, tissues sections were deparaffinized blocked with sniper (Biocare Medical) for 5 minutes and stained using Vector ABC or ARK (DAB) kits with 2 hours incubation with the antibodies. The antibodies used include anti-human PECAM1, anti-human vWF (DAKO), and anti-human CD34 (Lab Vision Corporation).

Flow Cytometry.

For isolation of PECAM1 positive cells, EBs at day 13 were dissociated with 0.025%/0.01% trypsin/EDTA, washed with PBS containing 5% FBS and incubated for 30 min with fluorescent-labeled PECAM1 antibodies (PharMingen, 30884X) on ice. Fluorescent-labeled cells were isolated using a flow cytometry cell sorter (FACStar, Becton Dickinson) and plated on 1% gelatin coated plates with endothelial cell growth medium (Clonetics). For analysis of endothelial cell markers, PECAM1+ cells grown in culture for 6 passages and HUVEC cells were dissociated using cell dissociation buffer (Gibco-BRL) washed with PBS containing 5% FBS. The cells were incubated with either isotype control (mouse IgG1 κ, PharMingen) or antigen specific antibodies: PECAM1-FITC (PharMingen), CD34-FITC (Miltenyi Biotec, AC136) and Flk-1/VEGFR-2-PE (ImClone Systems). Cells were analyzed live (without fixation) by using propidium iodide to exclude dead cells on a FACScan (Becton Dickinson) with CELLQUEST software.

Electron Microscopy

Cell seeded in Matrigel-coated 35 mm plates were fixed for one hour in 2.5% gluteraldehyde, 3% paraformaldehyde and 7.5% sucrose in 0.1 M sodium cacodylate buffer (pH 7.4) and then post fixed in 1% OsO₄ in veronal-acetate buffer for 1 hour. The cells were stained en bloc overnight with 0.5% uranyl acetate in veronal-acetate buffer (pH 6.0), dehydrated and embedded in Spurrs resin. Sections were cut on a Reichert Ultra cut E at a thickness of 70 nm using a diamond knife. Sections were examined using a Phillips EM410.

Biodegradable Polymer Matrix

Porous sponges composed of poly-L-lactic acid (PLLA) and poly-lactic-glycolic acid (PLGA) were fabricated mainly as previously described (Mooney, et al., (1997) J Biomed Mater Res 37, 4130-20). Briefly, PLLA (Polysciences) and PLGA (Boehringer Ingelheim) 1:1 were dissolved in chloroform to yield a solution of 5% polymer (w/v), and 0.24 ml of this solution was loaded into molds packed with 0.4 gr of sodium chloride particles. The solvent was allowed to evaporate and the sponges subsequently immersed for 8 hours in distilled water (changed every hour) to leach the salt and create an interconnected pore structure. The sponges, which had an average pore diameter of 250 μm, were cut to 0.5×4×5 mm. Before transplantation, sponges were soaked in 70% EtOH over night and washed three times with PBS.

Transplantation into SCID Mice

PECAM1+ cells (1×10⁶) were resuspended in 50 μl of 1:1 mix of culture medium and Matrigel (BD Biosciences) and allowed to absorb into the PLLA/PLGA polymer sponges. After 30 min incubation in 37° C., to allow for gelation of Matrigel, the cells+scaffolds were implanted subcutaneously in the dorsal region of 4 weeks old SCID mice (CB.17.SCID Taconic). 7 or 14 days after transplantation, the implants were retrieved, fixed overnight in 10% buffered Formalin at 4° C., embedded in Paraffin and sectioned for histological examination.

Results

Endothelial gene expression during hEB differentiation. To isolate endothelial cells from human embryonic stem (hES) cells, we first characterized their vasculogenic potential by analyzing the expression of endothelial specific genes and proteins during hES differentiation. Spontaneous in vitro differentiation of H9 hES cells into endothelial cells was investigated after removing undifferentiated cells from their mouse embryonic fibroblast (MEF) feeder layer and placing them into petri dishes with culture medium lacking leukemia inhibitor factor (LIF) and basic fibroblast growth factor (bFGF) for induction of EB formation (Thomson, et al., (1998) Science 282, 1145-1147). At different time points during the differentiation process the cultured hEBs were collected and RNA was extracted for analysis of endothelial-related gene expression using RT-PCR. The genes analyzed included endothelial cell adhesion molecules such as platelet endothelial cell adhesion molecule-1(PECAM1/CD31), vascular endothelial-cadherin (VE-cad) and CD34; growth factor receptors such as vascular endothelial growth factor receptor (Flk-1/KDR/VEGFR-2), and Tie-2; transcription factors GATA-2 and GATA-3; and AC133/CD133, a cell surface marker of vascular/hematopoietic stem and progenitor cells (DeLisser, et al., (1994) Immunol Today 15, 490-5; Lampugnani (1992); Young, et al., (1995) Blood 85, 96-105; Yamaguchi, et al., (1993) Development 118, 489-98; Sato, et al., (1993) Proc Natl Acad Sci USA 90, 9355-8; Weiss, et al., (1995) Exp Hematol 23, 99-107; Peichev, et al., (2000) Blood 95, 952-8).

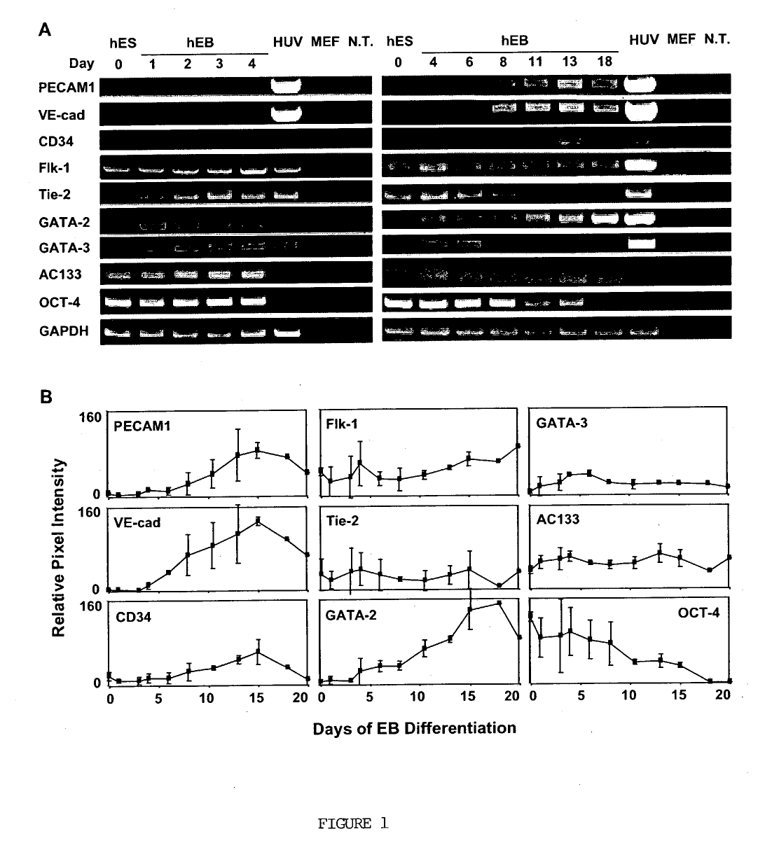

As shown in FIG. 1, the levels of endothelial markers PECAM1, VE-cad and CD34 increased during EB differentiation, reaching a maximum at days 13-15 and indicating a differentiation process toward endothelial cells. GATA-2 was expressed earlier and rose dramatically toward day 18. Unlike the mouse system, the VEGF receptor -Flk-1- is expressed in undifferentiated cells (also reported recently by Kaufman et al 2001 in H1 line), and increased very slightly during differentiation (Kaufman, et al., (2001) Proc Natl Acad Sci USA 98, 10716-21). The tyrosine kinase receptor Tie-2 and the transcription factors GATA-3 are also expressed in hES cells and their expression increased during the first six days of EB differentiation and then decreased (FIGS. 1, A and B). AC133 is expressed in undifferentiated cells as well as in differentiated EB cells in a pattern similar to that of Flk-1. The levels of Oct-4, which is known to be expressed in undifferentiated cells, served as a control (Yeom, et al., (1996) Development 122, 881-94). Oct-4 expression shows the undifferentiated stage of the cells at day 0 as it is expressed in the cells in high levels. Oct-4 expression subsequently goes down, indicating that the differentiation process is proceeding in the EBs. Human umbilical vein endothelial (HUVEC) cells were used as a positive control for the expression of the various human endothelial genes. The MEF feeder layer cells were used as a negative control, and did not express any of the human specific genes examined. These data demonstrate an increase in expression of several endothelial cell genes during EB differentiation reaching a maximum at days 13-15 (FIGS. 1, A and B). Some genes were expressed in the undifferentiated cells in either high levels (Flk-1, AC133, Tie-2) or lower levels (GATA-3, CD34), and others became notable following EB formation and differentiation (PECAM1, VE-cad, GATA-2) (FIGS. 1, A and B).

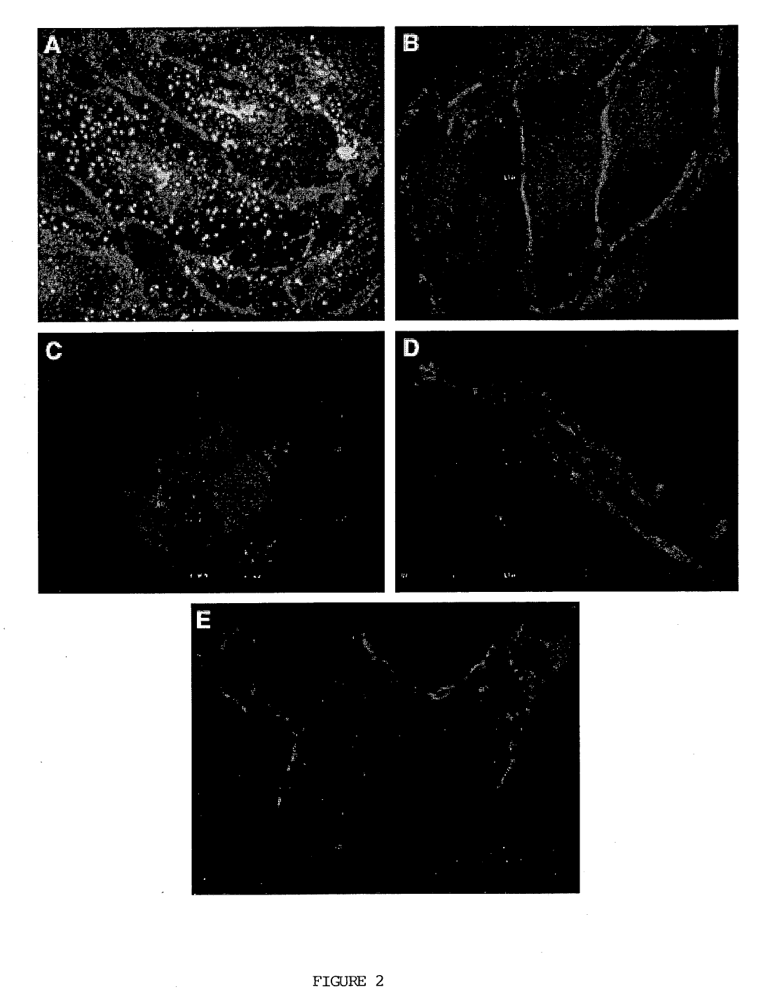

Formation of vessel-like structure in differentiating hEBs. Analysis of endothelial specific protein expression in day 13 EBs indicated that all EBs had defined cell areas expressing PECAM1 (FIG. 2C). Further analysis of PECAM1 positive cells, with various endothelial specific proteins, indicated these cells are endothelial-like, expressing PECAM1 and VE-cad adhesion molecules at cell-cell adhesion sites and von Willebrand Factor (vWF) in large granules dispersed throughout the cytoplasm (FIGS. 2, A and B). Within these EBs, the endothelial cells were not found as single cells but in groups organized in specific channel-like structures (FIGS. 2, D and E), showing that hES cells cultivated as EBs spontaneously differentiate to endothelial cells and blood vessel-like structures.

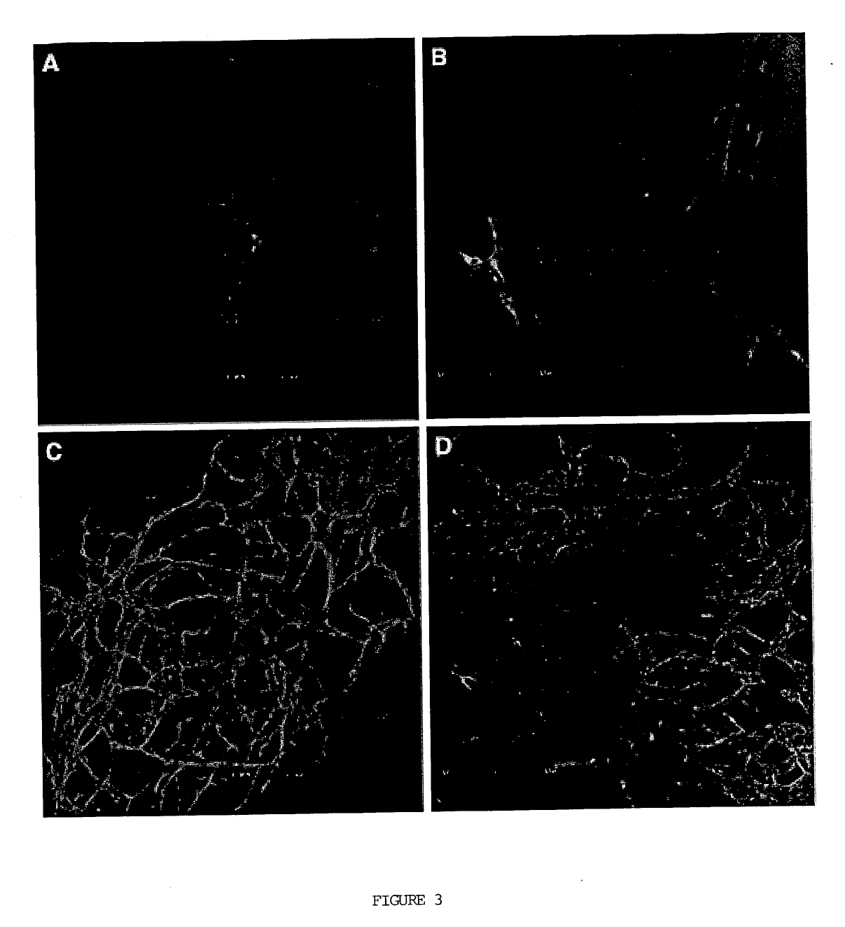

To further study this vascularization-like process, EBs at different time points were stained with PECAM1 antibodies and analyzed using confocal microscopy. FIG. 3 demonstrates that the capillary area increased during subsequent maturation steps up to day 13. On day 4, PECAM1-positive cells were observed in a low percentage of the EBs and concentrated in small cell clusters (FIG. 3A). From day 6 on, some sprouting of endothelial structures that resembled capillaries became evident (FIG. 3B). From day 10 on, 100% of EBs contained extended areas of network-like capillaries structures (FIG. 3C). The positive area was larger at day 13 and the network structure became more complex (FIG. 3D). The time course of cell differentiation and the development of extensive vasculature-resembling structures within the EB correlates with the RT-PCR analysis which demonstrates the subsequent increase in RNA levels of the endothelial genes PECAM1, VE-cad, CD34, reaching a maximum between day 13-15 (FIG. 1).

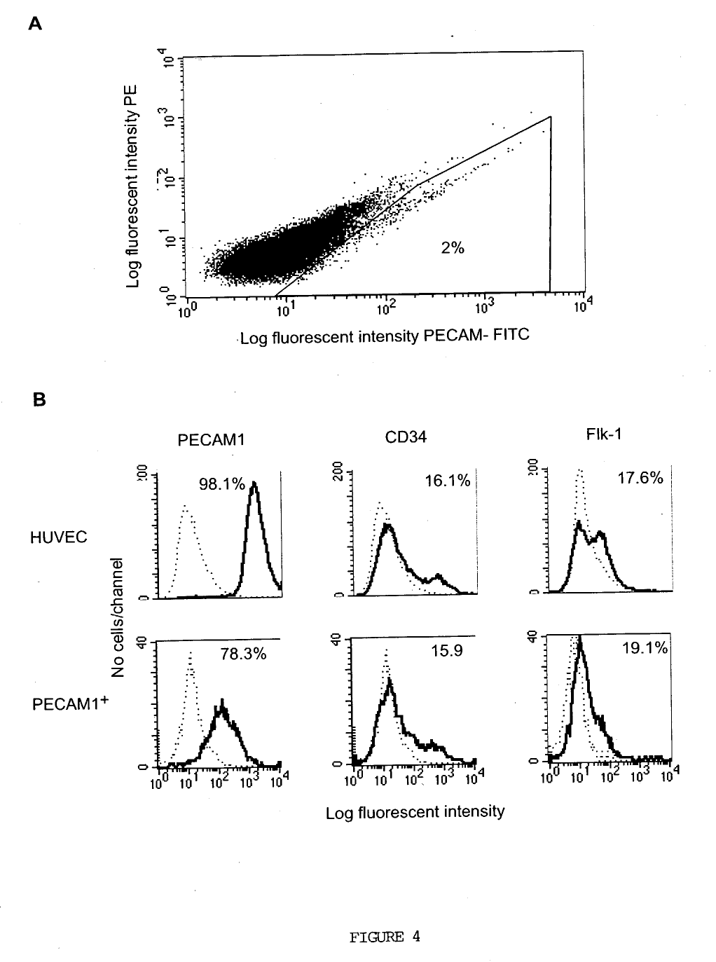

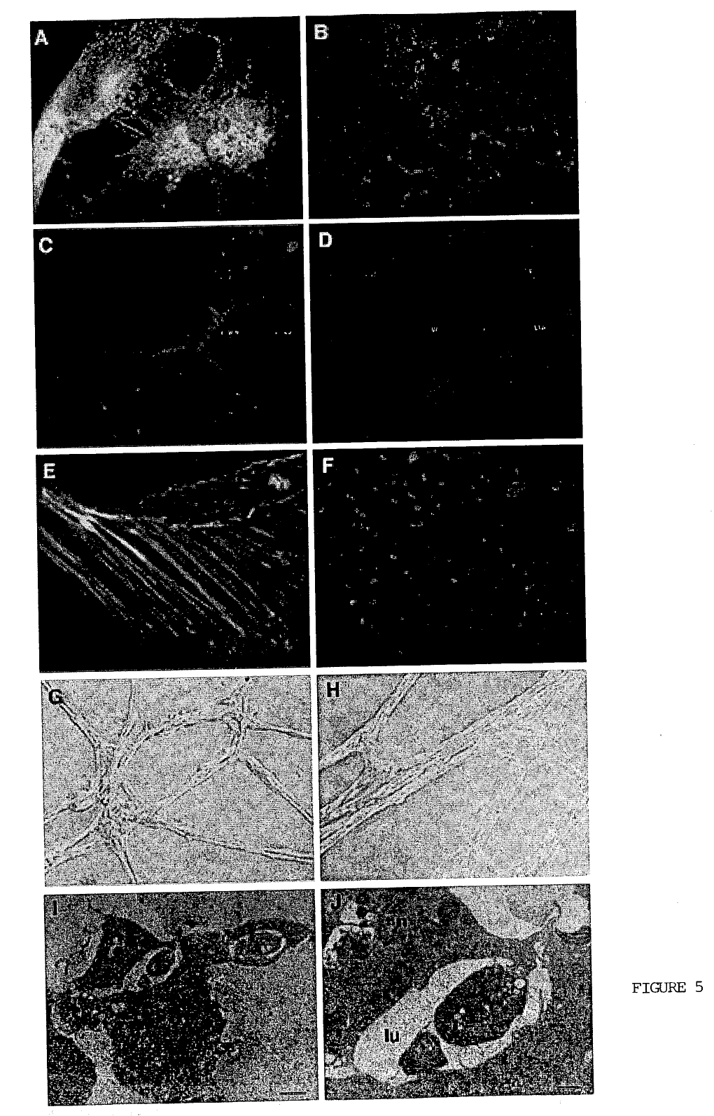

Endothelial cells derived from hEBs. Based on the analysis of endothelial gene and protein expression, we determined the method and time point in which to isolate human embryonic endothelial cells. We decided to use antibodies against PECAM1 for the isolation, as PECAM1 has been shown as the definitive marker for mouse embryonic endothelial cells, and in human EBs is expressed in vessel-like structures in correlation with VE-cad and vWF expression (FIGS. 2 and 3) suggesting that it could serve as a marker for human embryonic-endothelial cells as well (Vecchi, et al., (1994) Eur J Cell Biol 63, 247-54). EBs at day 13 were dissociated, stained with fluorescent-labeled anti-PECAM1 antibodies and the PECAM1 positive cells (2%) were sorted using flow cytometry (FIG. 4A). To confirm an endothelial-like phenotype of PECAM1+ cells grown in culture, we assayed them for the expression of endothelial cell markers. Isolated PECAM1+ cells (after several passages in culture) and HUVEC cells were incubated with fluorescent-labeled antibodies and analyzed by FACS. FIG. 4B shows that the expression profile of CD34 and Flk-1 in isolated PECAM1+ cells is similar to the HUVEC cells. Expression of PECAM1 is also comparable but with higher expression in the HUVEC cells (98%) compared to PECAM1+ isolated cells (78%). In addition to FACS analysis, we studied the distribution of adhesion molecules by immunofluorescence microscopy. PECAM1+ cells appear to present a correct organization of endothelial junctions; N-cadherin and the endothelium-specific VE-cadherin are distributed at adherent type junctions (FIGS. 5, C and D), a class of cell adhesions characterized by their interaction with the actin microfilament system (Ayalon, et al., (1994) J Cell Biol 126, 247-58). Actin stress fibers are found throughout the cells and end in both the cell-cell adherence junctions and focal contacts as seen by double staining with vinculin (FIG. 5E). The tight junction component, PECAM1, is distributed at the intercellular clefts, and the endothelial marker vWF is highly expressed in the cytoplasm (FIGS. 5, A and B).

Take-up of ac-LDL has been used to characterize endothelial cells (Voyta, et al., (1984) J Cell Biol 99, 2034-40). To evaluate whether embryonic derived PECAM1+ cells are able to incorporate ac-LDL, cells were incubated with Dill-Ac-LDL and subsequently examined by fluorescence microscopy. As shown in FIG. 5F, embryonic derived PECAM1+ cells were brightly fluorescent whereas the fluorescent intensity of PECAM1-cells was at background levels.

The characteristics of human embryonic PECAM1+ cells were also assessed by culture in matrigel, an extracellular matrix basement membrane that can be used to promote differentiation of endothelial cells (Grant, et al., (1991) In Vitro Cell Dev Biol 27A, 327-36). When PECAM1+ cells were cultured on matrigel they were able to spontaneously reorganize in cord-like structures when maintained in culture for several days (FIGS. 5, G and H). Electron microscopy analysis of the cord cross section indicated that the cords have a lumen (FIGS. 5 I-J) suggesting that the cells have the capacity to differentiate and form tube-like structures under suitable conditions.

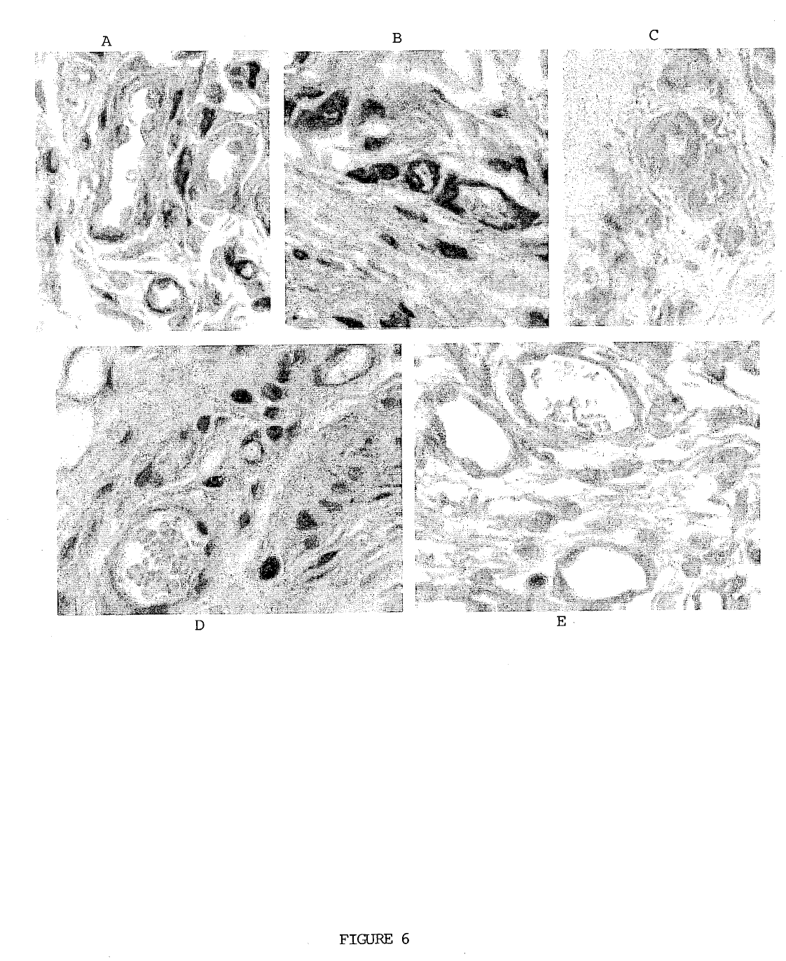

Transplantation of PECAM+ cells into SCID mice. To analyze the therapeutic potential of hES derived endothelial cells, we studied their behavior in vivo. The cells were seeded on highly porous PLLA/PLGA biodegradable polymer scaffolds, commonly used as scaffolds for tissue engineering (Putnam, et al., (1996) Nat Med 2, 824-6). Sponges seeded with embryonic derived PECAM+ cells were implanted in the subcutaneous tissue of SCID mice. At the time of implant retrieval (up to 14 days), no signs of infection were detected and inflammation was minimal. Implants maintained in mice for at least 7 days became encapsulated by fibrous connective tissue that was permeated by mouse blood vessels. Histological examination using antibodies that are human specific and do not react with mice microvessels, show microvessels that are immunoreactive with human PECAM1 and CD34 (FIGS. 6, A-E). Some of these human-positive vessels had mouse blood cells in their lumen suggesting that microvessels had formed and anastomosed with the mouse vasculature, becoming functional blood-carrying microvessels.

Discussion

This study indicates that human ES cells, when induced to form EBs, can spontaneously differentiate into the endothelial lineage, ultimately forming vascular structures. Our data demonstrate an increase in expression of several endothelial cell genes during EB differentiation reaching a maximum at days 13-15. Some genes were expressed in undifferentiated cells in either high levels (Flk-1, AC133, Tie-2) or lower levels (GATA-3, CD34), and others became notable following EB formation and differentiation (PECAM1, VE-cad, GATA-2). In the mouse, these genes are not expressed in ES (or expressed in very low levels that disappear by day 1 as EB are formed (PECAM1, Tie-2)) and start to appear only around day 3 and later. (Flk-1 at day 2-3, PECAM and Tie-2 at day 4, VE-cad and Tie-1 at day 5) (Vittet (1996); Robertson, et al., (2000) Development 127, 2447-59). Mouse and human ES cells differ in morphology, population doubling time, and growth factor requirements. Undifferentiated mouse cells, for example, can be maintained as undifferentiated cells independent of feeder layer if growth factors such as LIT are added to the media (Matsuda, et al., (1999) Embo J 18, 4261-9). However, human cells will differentiate if grown without feeder layer or feeder layer conditioned medium even in the presence of LIF (Thompson (1998); Xu, et al., (2001) Nat Biotechnol 19, 971-4). Thus, different mechanisms of response to LIF, and LIF removal between mouse and human ES cells may affect differences in gene expression patterns observed in the transition from the undifferentiated to the differentiated stage of the cells. It is possible that gene expression of endothelial markers in undifferentiated hES cells can be related to “escape” of some cells from the undifferentiated stage of hES cells or due to different basic definitions (regarding gene expression) of the undifferentiated state of hES cells kept in current culture conditions. However due to significant differences between early human and mouse development, and differences in behavior of mouse and human ES cells, the pattern of human endothelial gene expression shown here might indicate differences in mechanism of embryonic endothelial differentiation. Our preliminary results indicate that growth factor cocktails (including bFGF and VEGF) known to induce endothelial differentiation in mice EBs do not have the same effect on hEBs (data not shown), pointing again to potential differences in the molecular mechanism underlying this process between the two systems, and emphasizing the need to analyze developmental processes using human systems.

The assembly of developing vascular-like structures could be observed during EBs outgrowth, as soon as the cells acquired the set of endothelial markers. The data also indicate that the capillary area in the EBs increased during subsequent maturation steps up to day 13 starting from cell clusters that later sprout into capillary-like structures and eventually become organized in a network-like arrangement. The increase in RNA expression of PECAM1, CD34, VE-cad and GATA-2 genes during EB differentiation correlates with the observed increase in number of endothelial cells expressing PECAM1 and VE-cad proteins as demonstrated by antibody staining of differentiating EBs (FIGS. 2 and 3). Antibody staining also indicates that at different stages of maturation, most markers appear to be coexpressed by the same cells. These data demonstrate for the first time that human ES cells, similar to mice ES cells, can spontaneously differentiate and organize in vitro in vessel-like structures in a pattern that resembles embryonic vascularization.

In the present study we isolated and maintained in culture endothelial cells derived from hES cells differentiated in vitro. PECAM1 antibodies have been used in the mouse system for isolation of endothelial cells (Balconi, et al., (2000) Arterioscler Thromb Vasc Biol 20, 1443-51). This procedure to obtain a pure culture of endothelial cells from ES allowed us to culture high numbers of human embryonic endothelial cells that can be grown in culture without losing endothelial characteristics.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

what is claimed is:

1. A population of embryonic endothelial cells produced in vitro from human embryonic stem cells.

2. The population of claim 1, wherein the embryonic endothelial cells are vasculogenic.

3. The population of claim 1, wherein at least 45% of the embryonic endothelial cells express one or more of platelet endothelial cell adhesion molecule-1 (PECAM1), GATA-2, N-cadherin (N-cad), vascular endothelial N-cadherin (VE-cad), and von Willebrand factor (vWF).

4. The population of claim 3, wherein at least 55% of the embryonic endothelial cells express one or more of PECAM1, GATA-2, N-cad, VE-cad, and vWF.

5. The population of claim 4, wherein at least 65% of the embryonic endothelial cells express one or more of PECAM1, GATA-2, N-cad, VE-cad, and vWF.

6. The population of claim 5, wherein at least 75% of the embryonic endothelial cells express one or more of PECAM1, GATA-2, N-cad, VE-cad, and vWF.

7. The population of claim 6, wherein at least 85% of the embryonic endothelial cells express one or more of PECAM1, GATA-2, N-cad, VE-cad, and vWF.

8. The population of claim 7, wherein at least 95% of the embryonic endothelial cells express one or more of PECAM1, GATA-2, N-cad, VE-cad, and vWF.

9. The population of claim 1, wherein at least 45% of the embryonic endothelial cells incorporate ac-LDL.

10. The population of claim 9, wherein at least 55% of the embryonic endothelial cells incorporate ac-LDL.

11. The population of claim 10, wherein at least 65% of the embryonic endothelial cells incorporate ac-LDL.

12. The population of claim 11, wherein at least 75% of the embryonic endothelial cells incorporate ac-LDL.

13. The population of claim 12, wherein at least 85% of the embryonic endothelial cells incorporate ac-LDL.

14. The population of claim 13, wherein at least 95% of the embryonic endothelial cells incorporate ac-LDL.

15. The population of claim 1, wherein at least 10% of the embryonic endothelial cells express CD34.

16. The population of claim 15, wherein at least 12% of the embryonic endothelial cells express CD34.

17. The population of claim 16, wherein at least 14% of the embryonic endothelial cells express CD34.

18. The population of claim 17, wherein at least 16% of the embryonic endothelial cells express CD34.

19. The population of claim 18, wherein at least 18% of the embryonic endothelial cells express CD34.

20. The population of claim 19, wherein at least 20% of the embryonic endothelial cells express CD34.

21. A tissue engineering construct comprising:
a cell support substrate; and
human embryonic endothelial cells supported by the cell support substrate.

22. The tissue engineering construct of claim 21, wherein the human embryonic endothelial cells are vasculogenic.

23. The tissue engineering construct of claim 21, wherein at least 45% of the human embryonic endothelial cells express one or more of PECAM1, GATA-2, N-cad, VE-cad, and vWF.

24. The tissue engineering construct of claim 23, wherein at least 55% of the human embryonic endothelial cells express one or more of PECAM1, GATA-2, N-cad, VE-cad, and vWF.

25. The tissue engineering construct of claim 24, wherein at least 65% of the human embryonic endothelial cells express one or more of PECAM1, GATA-2, N-cad, VE-cad, and vWF.

26. The tissue engineering construct of claim 25, wherein at least 75% of the human embryonic endothelial cells express one or more of PECAM1, GATA-2, N-cad, VE-cad, and vWF.

27. The tissue engineering construct of claim 26, wherein at least 85% of the human embryonic endothelial cells express one or more of PECAM1, GATA-2, N-cad, VE-cad, and vWF.

28. The tissue engineering construct of claim 27, wherein at least 95% of the human embryonic endothelial cells express one or more of PECAM1, GATA-2, N-cad, VE-cad, and vWF.

29. The tissue engineering construct of claim 21, wherein at least 10% of the human embryonic endothelial cells express CD34.

30. The tissue engineering construct of claim 29, wherein at least 12% of the human embryonic endothelial cells express CD34.

31. The tissue engineering construct of claim 30, wherein at least 14% of the human embryonic endothelial cells express CD34.

32. The tissue engineering construct of claim 31, wherein at least 16% of the human embryonic endothelial cells express CD34.

33. The tissue engineering construct of claim 32, wherein at least 18% of the human embryonic endothelial cells express CD34.

34. The tissue engineering construct of claim 21, wherein at least 45% of the human embryonic endothelial cells incorporate ac-LDL.

35. The tissue engineering construct of claim 34, wherein at least 55% of the human embryonic endothelial cells incorporate ac-LDL.

36. The tissue engineering construct of claim 35, wherein at least 65% of the human embryonic endothelial cells incorporate ac-LDL.

37. The tissue engineering construct of claim 36, wherein at least 75% of the human embryonic endothelial cells incorporate ac-LDL.

38. The tissue engineering construct of claim 37, wherein at least 85% of the human embryonic endothelial cells incorporate ac-LDL.

39. The tissue engineering construct of claim 38, wherein at least 95% of the human embryonic endothelial cells incorporate ac-LDL.

40. The tissue engineering construct of claim 21, further comprising a polymer matrix infused with the cell support substrate.

41. The tissue engineering construct of claim 40, wherein the polymer matrix comprises poly(glycolic acid), collagen-glycosaminoglycan, collagen, poly(lactic acid), poly(lactic-co-glycolic acid), poly(anhydride), poly(hydroxy acid), poly(orthoester), poly(propylfumerate), polysaccharide, polypyrrole, polyaniline, polythiophene, polystyrene, polyester, polyurethane, polyurea, poly(ethylene vinyl acetate), polypropylene, polymethacrylate, polyethylene, poly(ethylene oxide), poly(carbonate), and any combination thereof.

42. The tissue engineering construct of claim 40, wherein the polymer matrix has a shape selected from particles, tube, sponge, sphere, strand, coiled strand, capillary network, film, fiber, mesh, and sheet.

43. The tissue engineering construct of claim 40, wherein the polymer matrix comprises a growth factor attached to the polymer via a member of a covalent and a non-covalent interaction.

44. The tissue engineering construct of claim 21, wherein the cell support substrate comprises a gel.

45. The tissue engineering construct of claim 44, wherein the gel comprises one or more of MATRIGEL™ and collagen-GAG.

46. The tissue engineering construct of claim 44, wherein the gel further comprises a member of the group consisting of collagen I, collagen IV, laminin, fibrin, fibronectin, proteoglycans, glycoproteins, glycoaminoglycans, proteinases, collagenases, chemotactic agents, growth factors, and any combination of the above.

47. The tissue engineering construct of claim 21, further comprising a liquid carrier mixed with the cell support substrate.

48. The tissue engineering construct of claim 21, further comprising at least one additional cell type.

49. The tissue engineering construct of claim 48, wherein the ratio between the embryonic endothelial cells and the additional cell type is at least 1:9.

50. The tissue engineering construct of claim 49, wherein the ratio between the embryonic endothelial cells and the additional cell type is at least 2.5:7.5.

51. The tissue engineering construct of claim 50, wherein the ratio between the embryonic endothelial cells and the additional cell type is at least 1:1.

52. The tissue engineering construct of claim 51, wherein the ratio between the embryonic endothelial cells and the additional cell type is at least 7.5:2.5.

53. The tissue engineering construct of claim 52, wherein the ratio between the embryonic endothelial cells and the additional cell type is at least 9:1.

54. The tissue engineering construct of claim 48, wherein the ratio between the additional cell type and the embryonic endothelial cells is at least 9:1.

55. The tissue engineering construct of claim 48, wherein the cell type is selected from muscle cell, nerve cell, connective tissue cell, or stem cell.

56. The tissue engineering construct of claim 21, wherein the cell support substrate is a tube and the embryonic endothelial cells are disposed on an inner surface of the tube.

57. The tissue engineering construct of claim 56, wherein the tube is a member of a decellularized blood vessel, a synthetic polymer tube, and a collagen tube.

58. A method of producing vasculogenic human cells in vitro, comprising:
providing a population of human embryonic stem cells;
culturing the stem cells in the absence of LIF and bFGF to stimulate formation of embryoid bodies containing the cultured stem cells; and
isolating PECAM1 positive cells from the embryoid bodies.

59. The method of claim 58, wherein the step of isolating comprises:
dissociating the embryoid bodies to separate the cultured stem cells;
incubating the cultured stem cells with a labeled PECAM1 antibody to distinguish the portion of the cultured stem cells that are PECAM1+; and
separating the PECAM1+ cells from the remaining cultured stem cells.

60. The method of claim 59, wherein the label is a member of a magnetic moiety and a fluorescent moiety.

61. The method of claim 58, wherein the step of providing comprises:
incubating a population of human embryonic stem cells in a culture medium; and
at least partially disaggregating the cultured stem cells.

62. A method of stimulating vasculogenesis in vivo, comprising:
performing the method of claim 58;
suspending the isolated PECAM1+ cells in a member of a liquid carrier, a cell support substrate, and a mixture of both; and
delivering the cell suspension to a tissue in an animal.

63. The method of claim 62, further comprising infusing a polymer matrix with the cell suspension before the step of inserting, wherein the step of inserting comprises implanting the polymer matrix into an animal.

64. The method of claim 62 or 63, wherein the cell support substrate comprises a gel.

65. The method of claim 64, wherein the gel comprises one or more of MATRIGEL™ and collagen-GAG.

66. The method of claim 64, wherein the gel further comprises a member of the group consisting of collagen I, collagen IV, laminin, fibrin, fibronectin, proteoglycans, glycoproteins, glycoaminoglycans, proteinases, collagenases, chemotactic agents, growth factors, and any combination of the above.

67. The method of claim 64, wherein the method further comprises allowing the gel to harden.

68. The method of claim 63, wherein the polymer matrix has a shape selected from particles, tube, sponge, sphere, strand, coiled strand, capillary network, film, fiber, mesh, and sheet.

69. The method of claim 63, wherein the step of delivering comprises disposing the polymer matrix about the outside of a blood vessel.

70. The method of claim 63, wherein the polymer matrix comprises a growth factor.

71. The method of claim 70, wherein the growth factor is selected from epidermal growth factor, bone morphogenetic protein, TGFβ, hepatocyte growth factor, platelet-derived growth factor, TGFα, IGF-I and II, hematopoetic growth factors, heparin binding growth factor, peptide growth factors, and basic and acidic fibroblast growth factors, nerve growth factor (NGF), vascular endothelial-derived growth factor (VEGF), and muscle morphogenic factor (MMP).

72. The method of claim 62, further comprising depositing the cell suspension on the inner surface of a tube.

73. The method of claim 72, wherein the tube is selected from a member of a collagen tube, a synthetic polymer, and a decellularized blood vessel.

74. The method of claim 63 or 73, further comprising allowing the cells to proliferate before the step of delivering.

75. The method of claim 74, further comprising permitting the cells to form a vascular structure during the step of allowing.

76. The method of claim 74, further comprising imparting a mechanical force on the cells during the step of allowing.

77. The method of claim 76, wherein the mechanical force is cyclic.

78. The method of claim 76, wherein the mechanical force is selected from the group consisting of hoop stress, shear stress, hydrostatic stress, compressive stress, and tensile stress.

79. The method of claim 62, wherein the tissue is ischemic.

80. The method of claim 62, wherein the tissue is selected from the group consisting of connective tissue, muscle tissue, nerve tissue, and organ tissue.

81. The method of claim 62, wherein the step of delivering comprises depositing the cells on the inner surface of a blood vessel.

82. The method of claim 62, wherein the cell support matrix includes a growth factor.

83. The method of claim 82, wherein the growth factor is selected from epidermal growth factor, bone morphogenetic protein, TGFβ, hepatocyte growth factor, platelet-derived growth factor, TGFα, IGF-I and II, hematopoetic growth factors, heparin binding growth factor, peptide growth factors, and basic and acidic fibroblast growth factors, nerve growth factor (NGF), vascular endothelial-derived growth factor (VEGF), and muscle morphogenic factor (MMP).

84. The method of claim 62, further comprising combining an additional cell type with the embryonic endothelial cells.

85. The method of claim 84, wherein the ratio of the additional cell type and the embryonic endothelial cells is between 1:9 and 9:1.

86. The method of claim 84, wherein the ratio of the additional cell type and the embryonic endothelial cells is greater than 9:1.

87. The method of claim 84, wherein the ratio of the embryonic endothelial cells to the additional cell type is greater than 9:1.

88. The method of claim 84, wherein the cells are selected from connective tissue cells, nerve cells, organ cells, muscle cells, and stem cells.

89. A method of producing a vascular structure, comprising:
performing the method of claim 58;
suspending the isolated PECAM1+ cells in a member of a liquid carrier, a cell support substrate, and a mixture of both;
infusing a polymer matrix with the cell suspension; and
allowing the PECAM+ cells to proliferate on the polymer matrix.

90. The method of claim 89, wherein the polymer matrix has a shape selected from particles, tube, sponge, sphere, strand, coiled strand, capillary network, film, fiber, mesh, and sheet.

91. The method of claim 89, wherein the cell support substrate comprises a gel.

92. The method of claim 91, wherein the gel comprises one or more of MATRIGEL™ and collagen-GAG.

93. The method of claim 92, wherein the gel further comprises a member of the group consisting of collagen I, collagen IV, laminin, fibrin, fibronectin, proteoglycans, glycoproteins, glycoaminoglycans, proteinases, collagenases, chemotactic agents, growth factors, and any combination of the above.

94. The method of claim claim 91, wherein the method further comprises allowing the gel to harden.

95. The method of claim 89, further comprising imposing a mechanical force on the matrix during the step of allowing.

96. The method of claim 92, wherein the mechanical force is cyclic.

97. The method of claim 92, wherein the mechanical force is selected from the group consisting of hoop stress, shear stress, hydrostatic stress, compressive stress, and tensile stress.

98. A method of producing vasculogenic human cells in vitro, comprising:
providing a population of human embryonic stem cells;
culturing the stem cells in the absence of LIS and bFGF to stimulate formation of embryoid bodies containing the cultured stem cells; and
isolating from the embryoid bodies cells that are positive for one or more of GATA-2, N-cad, VE-cad, and vWF.

99. The method of claim 98, wherein the step of isolating comprises:
dissociating the embryoid bodies to separate the cultured stem cells;
incubating the cultured stem cells with labeled antibodies for one or more of GATA-2, N-cad, VE-cad, and vWF; and
separating cells that express one or more of GATA-2, N-cad, VE-cad, and vWF from the remaining cultured stem cells.

100. A method of stimulating vasculogenesis in vivo, comprising:
performing the method of claim 98;
suspending the isolated cells in a member of a liquid carrier, a cell support substrate, and a mixture of both; and
delivering the cell suspension to a tissue in an animal.

101. A method of producing a vascular structure, comprising:
performing the method of claim 97;
suspending the isolated cells in a member of a liquid carrier, a cell support substrate, and a mixture of both;
infusing a polymer matrix with the cell suspension; and
allowing the isolated cells to proliferate on the polymer matrix.
